(19) United States
(12) Patent Application Publication
(10) Pub. No.: US 2008/0292700 A1
(43) Pub. Date: Nov. 27, 2008

(54) CONTROLLED RELEASE FORMULATIONS USING INTELLIGENT POLYMERS

(75) Inventors: Tien NGHIEM, Lucan (IE); Graham Jackson, Celbridge (IE)

Correspondence Address: OBLON, SPIVAK, MCCLELLAND MAIER & NEUSTADT, P.C., 1940 DUKE STREET, ALEXANDRIA, VA, 22314, US

(73) Assignee: BIOVAIL LABORATORIES, St. Michael (BB)

(21) Appl. No.: 12/186,853

(22) Filed: Aug. 6, 2008

Related U.S. Application Data
(63) Continuation-in-part of application No. 11/109,067, filed on Apr. 19, 2005.
Application No. 11/109,067 is a continuation of application No. 09/403,437, filed on Dec. 20, 1999, now Pat. No. 6,893,661.
(60) Provisional application No. 60/036,551, filed on Apr. 21, 1997.

(51) Int. Cl. A61K 9/22; A61P 25/08; A61P 25/06; A61P 3/04; A61P 25/32; A61P 25/36; A61P 25/34; A61P 25/18

(52) U.S. Cl. 424/468

(57) ABSTRACT

A controlled release pharmaceutical composition comprises (a) topiramate or a pharmaceutically acceptable salt thereof, (b) a first intelligent polymer component; and (c) a second intelligent polymer component having opposite wettability characteristics to the first intelligent polymer component. The polymer components are effective for controlled release of the pharmaceutically active substance from the composition.

### CROSS-REFERENCE TO RELATED APPLICATIONS

This application is a continuation-in-part of U.S. application Ser. No. 11/109,067, filed Apr. 19, 2005, which is a continuation of U.S. application Ser. No. 09/403,437, filed Dec. 20, 1999, now Pat. No. 6,893,661, filed as 371 of international application PCT/CA98/00274, filed Apr. 3, 1998, which claims priority to U.S. provisional application 60/036,551, filed Apr. 21, 1997; each of which is incorporated herein by reference in its entirety.

### FIELD OF THE INVENTION

The present invention is directed to novel controlled release formulations of pharmaceutically active substances and methods for their preparation. More particularly, the present invention relates to an easily absorbable, controlled release pharmaceutical formulation utilizing groups of intelligent polymers having opposing wettability characteristics. The present invention also relates to a controlled release formulation of topiramate.

### BACKGROUND OF THE INVENTION

Controlled release formulations of pharmaceutical agents is an extremely large market in the pharmaceutical and medical fields. A number of types of controlled release dosage forms are known, including matrix tablet systems incorporating active ingredients, fillers and various types of excipients. The very different properties of numerous different types of pharmaceutically active ingredients has necessitated the development of a number of different drug delivery systems utilizing polymer technology in order to provide an appropriate release of a particular medicament after oral ingestion by a patient.

U.S. Pat. Nos. 4,601,894 and 4,657,757 describe a controlled release drug delivery system which contains hydroxypropylmethylcellulose (HPMC) and a second polymer such as ethylcellulose, methylcellulose, sodium carboxymethyl cellulose or other cellulose ethers. U.S. Pat. No. 4,680,323 describes a carrier system comprising hydroxypropyl cellulose and a carboxy vinyl polymer. U.S. Pat. No. 4,695,591 describes the use of HPMC for mediating controlled release of pharmaceutically active substances. U.S. Pat. No. 4,994,276 teaches a free-flowing directly compressible granulation useful as a slow release pharmaceutical excipient. The excipient includes a hydrophilic matrix which includes a heteropolysaccharide and a polysaccharide material capable of cross-linking the heteropolysaccharide.

U.S. Pat. No. 4,167,558 teaches a novel sustained release tableted formulation for oral administration. The formulation is hydrodynamically balanced to be buoyant in gastric juice thereby remaining in the stomach for an extended period of time. U.S. Pat. No. 4,259,314 teaches a method and composition for the preparation of controlled long-acting pharmaceuticals using a dry carrier or base material comprising an effective amount of hydroxypropyl methylcellulose and hydroxypropyl cellulose suitable for use with both hygroscopic and non-hygroscopic materials. The controlled long-acting products of the invention are suitable for use in the form of lozenges, buccal tablets, oral tablets or suppositories.

U.S. Pat. No. 4,308,251 teaches a tablet formulation comprising an effective amount of an active acidic therapeutic agent, a release-controlling agent and an erosion-promoting agent in relative amounts to provide a criticality factor of less than 450, and in proportions of release-controlling and erosion-promoting agent, respectively, between 0.8-1.6 and 1.0-7.5 weight percent per tablet. The tablets of this invention exhibit zero order release in vitro and closely approximate zero order absorption in vivo.

U.S. Pat. No. 4,361,545 teaches a class of solid pharmaceutical formulations which provides slow, zero order in vivo release of a wide range of pharmaceutically active ingredients upon oral administration. A broad range of release rates can be preselected by suitable adjustments of tablet properties. The formulations are based upon control of active ingredient release from the surface of the tablet via a controlled surface erosion mechanism.

U.S. Pat. No. 4,389,393 teaches a carrier base material combined with a therapeutically active medicament and shaped and compressed to a solid unit dosage form having a regular and prolonged release pattern upon administration, the carrier base material being one or more hydroxypropylmethylcelluloses or a mixture of one or more hydroxypropylmethylcelluloses having a methoxy content of 16-24 weight %, a hydroxypropoxyl content of 4-32 weight % and an average molecular weight of at least 50,000.

U.S. Pat. No. 4,525,345 teaches a constant release rate indomethacin formulation in tablet unit dosage form containing an admixture of from 50 to 200 mg of indomethacin, from about 1.7 to 3.7 weight percent of a slow-dissolving, water-insoluble cellulose derivative, from about 1.5 to 5.0 weight percent of a tableting disintegrant, and from about 40 to 80 weight percent of a pharmaceutically acceptable bulking agent or diluent.

U.S. Pat. No. 4,556,678 teaches a tablet consisting essentially of a therapeutically effective amount of propranolol to provide a sustained release thereof over a prolonged period of time. The tablet comprises compressed granules having from about 0.1 to about 10 parts by weight hydroxypropyl methylcellulose and about one part by weight hydroxpropyl cellulose.

U.S. Pat. No. 4,692,337 teaches a sustained release pharmaceutical tablet comprising theophylline and ethyl cellulose uniformly dispersed therein in an amount of 5 to 200 parts by weight of ethyl cellulose based on 100 parts by weight of the theophylline.

U.S. Pat. No. 4,756,911 teaches a controlled release pharmaceutical formulation in the form of a coated tablet, containing a core portion from which medicament, such as procainamide hydrochloride, is slowly released over a controlled length of time. The core also includes one or more primary hydrocolloid gelling agents which is a hydropropymethyl cellulose having a viscosity of within the range of from about 1,000 to about 6,000 centipoises in 2% solution at 20° C., a methoxyl content of 28-30% and optionally a secondary hydrocarbon gelling agent, such as hydroxpropyl cellulose and/or methyl cellulose.

U.S. Pat. No. 5,073,380 teaches a pharmaceutical sustained release tablet containing a pharmaceutical active, hydroxyethyl cellulose, a wicking agent, povidone, pregelatinized starch, lubricant and a glidant.

U.S. Pat. No. 5,417,982 teaches a controlled release formulation for use with a variety of drugs or hormones in microspherical form. The drug or hormone, e.g. bovine somatropine, is suspended in a polymer matrix formed from at least two highly water soluble biodegradable polymers. The microspheres are coated with a (δ, 1 lactide-glycolide) copolymer.

U.S. Pat. No. 4,968,509 teaches an acetaminophen-sustained release tablet formed by making a wet granulation, using Povidone (PVP) in water or alcohol-water as the granulating fluid which is mixed with acetaminophen, hydroxyethyl cellulose, a wicking agent e.g. microcrystalline cellulose, then drying and milling the granulation and blending with dry powdered erosion promoter, e.g. pregelatinized starch, wicking agent, lubricant e.g. magnesium stearate and glidant e.g. silicon dioxide, and compressing the resultant granulation.

U.S. Pat. No. 5,462,747 teaches a pharmaceutical sustained release homogeneous tablet formed by making a wet granulation using povidone (PVP) in alcohol as the granulating fluid mixed with a pharmaceutical active, ethylcellulose, a wicking agent, e.g. microcrystalline cellulose, an erosion promoter, e.g. pregelatinized starch, then drying and milling the granulation and blending with a dry powdered erosion promotor, wicking agent, lubricant and glidant.

U.S. Pat. No. 5,543,154 teaches a device for the controlled delivery of a beneficial agent as a gelatinous dispersion consisting of a core which contains a beneficial agent, a polymer which forms gelatinous microscopic particles upon hydration and if desired an agent to modulate the hydration of the polymer; and an impermeable, insoluble coating which adheres to and surrounds the core and contains apertures which provides an area for the hydration and release of a disperson comprising gelatinous microscopic particles.

U.S. Pat. No. 5,439,687 teaches pharmaceutical dosage forms for the daily oral administration of nifedipine or of another calcium antagonist of the dihydropyridine type, characterised by the homogeneous matrix containing 2-50% by weight of hydroxypropylmethylcellulose having an average molecular weight of 20,000-250,000, 5-60% by weight of a calcium antagonist of the dihydropyridine type, as well as excipients compatible with the formulation.

U.S. Pat. No. 5,264,446 teaches a solid pharmaceutical composition of nifedipine crystals having specific surface area of 1-4 m² /g in the form of tablets, pills, dragees, capsules, suppositories, sachets or two layer tablets resulting in sustained release.

While these systems can provide for sustained release of a selected active ingredient, most of these systems have the disadvantage of being affected by the presence of food and gastrointestinal enzymes in the gastrointestinal (GI) tract. Therefore, the active ingredient is often not delivered in a consistent and reproducible manner. In addition, osmotic and press coated tablets are particularly difficult and expensive to manufacture.

It is therefore particularly desirable to design an efficient drug delivery system that is capable of controlled drug delivery of both high dose, highly soluble, hydrophillic and low dose, poorly soluble, hydrophobic pharmaceutically active substance(s) into the gastrointestinal tract (GIT) in order to provide sustained therapeutic effects for over 24 hours with only a single dose and without any food effect. It is also highly desirable to develop a drug delivery system that is relatively easy and inexpensive to manufacture and more efficient in providing a sustained release of pharmaceutical agents than the known controlled delivery systems.

Topiramate (2,3:4,5-bis-O-(1-methylethylidene)-β-D-fructopyranose sulfamate) is a monosaccharide sulfamate derivative with the chemical structure below.

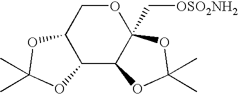

Topiramate is currently marketed in the United States for the treatment of seizures in epileptic patients and for the prevention of migraine headache as Topamax® (currently approved in the US under NDA #020505) and Topamax Sprinkle® (currently approved in the US under NDA #020844). Other disorders for which topiramate may be useful include but are not limited to obesity; alcohol, cocaine and/or tobacco dependence; bipolar disorder; and other central nervous system disorders. The solubility of topiramate in water at room temperature is relatively low (about 9.8 mg/mL), which presents a challenge for the formulation of topiramate as a once daily controlled release dosage form.

U.S. Pat. Nos. 6,559,293 and 6,699,840 describe salts of topiramate which have a higher aqueous solubility than topiramate itself, and which may be formulated as controlled and delayed release dosage forms. Controlled release preparations of topiramate are also described in WO 2008/027557, US 2007/0243254, WO 2006/009403, WO 2006/063078, US 2006/0034927, WO 2005/065648, WO 2005/065647, WO 2005/020959, US 2005/0287213, US 2005/0175690, US 2005/0169992, US 2005/0136108, US 2005/0129765, US 2005/0058707, US 2005/0013863, US 2004/0115262, and US 2003/0072802.

### SUMMARY OF THE INVENTION

An object of the present invention is to provide a novel controlled sustained release delivery composition which may contain a wide variety of pharmaceutically active ingredients and which demonstrates good absorbability of the selected active ingredient and a maintenance of the therapeutically effective blood level of the pharmaceutically active ingredient for a long duration of time by one time administration. This novel controlled release composition and system has been named intelliGITransporter™.

It is a further object of the present invention to provide a controlled release delivery composition wherein the selection of the pharmaceutically active ingredient, the physiochemical properties, the proportion of polymer blend and the wettability of the pharmaceutically active substance(s) provides effective controlled release of the pharmaceutically active substance(s).

It is yet a further object of the present invention to provide an effective drug delivery composition that is capable of controlled drug delivery of both high dose, highly soluble hydrophilic or low dose poorly soluble hydrophobic pharmaceutically active substance(s) to the gastrointestinal tract with a zero or first order kinetics.

An aspect of the present invention provides a novel controlled release delivery composition comprising at least one selected pharmaceutically active ingredient incorporated within a homogeneous matrix comprising effective amounts of two intelligent polymers having opposing wettability characteristics, wherein one polymer is selected which demonstrates a stronger tendency towards hydrophobicity and the other polymer is selected which demonstrates a stronger tendency towards hydrophilicity.

In at least one embodiment, the active pharmaceutical ingredient selected has a water contact angle (θ) such that cos θ is between +0.9848 and −0.9848. In at least one embodiment, the pharmaceutically active ingredient is topiramate or a pharmaceutically acceptable salt thereof.

According to at least one embodiment, the present invention provides a controlled release matrix tablet comprising:
(a) topiramate or a pharmaceutically acceptable salt thereof;
(b) a first intelligent polymer component; and
(c) a second intelligent polymer component having opposite wettability characteristics to said first intelligent polymer component;
   wherein the first intelligent polymer component is more hydrophobic than the second intelligent polymer component; and wherein the first and second intelligent polymer components constitute a substantially homogeneous matrix, wherein the topiramate or pharmaceutically acceptable salt thereof is substantially homogeneously dispersed in the substantially homogeneous matrix.

In at least one embodiment, the controlled release matrix tablet comprising topiramate or a pharmaceutically acceptable salt thereof exhibits an in vitro/in vivo correlation. In at least one embodiment, the controlled release matrix tablet comprising topiramate or a pharmaceutically acceptable salt thereof is bioequivalent to a reference formulation of topiramate or a pharmaceutically acceptable salt thereof. In at least one embodiment, the controlled release matrix tablet comprising topiramate or a pharmaceutically acceptable salt thereof exhibits an in vitro dissolution profile at pH 6.8 or at pH 4.5 such that after about 1 hour, no more than about 15% of the topiramate or pharmaceutically acceptable salt thereof is released; after about 2 hours, from about 5% to about 35% of the topiramate or pharmaceutically acceptable salt thereof is released; after about 4 hours, from about 20% to about 60% of the topiramate or pharmaceutically acceptable salt thereof is released; after about 8 hours, from about 35% to about 95% of the topiramate or pharmaceutically acceptable salt thereof is released; after about 12 hours, no less than about 50% of the topiramate or pharmaceutically acceptable salt thereof is released; and after about 16 hours, no less than about 60% of the topiramate or pharmaceutically acceptable salt thereof is released.

In at least one embodiment, the composition of the present invention comprises topiramate or a pharmaceutically acceptable salt thereof and exhibits a dissolution profile at pH 4.5 or at pH 6.8 characterized by the following equation:

y=100−100*e^{(-a*x}^{^{b}})

where:

y=% dissolution;

x=sampling time;

a=scale parameter which ranges from about 0.06 to about 0.12;

b=shape parameter which ranges from about 0.9 to about 1.6; and

100=the cumulative percentage of the topiramate released at time infinity.

In at least one embodiment, the intelligent polymer demonstrating a stronger tendency towards hydrophobicity is ethylcellulose (EC) whereas the intelligent polymer demonstrating a stronger tendency towards hydrophilicity is hydroxyethylcellulose (HEC) and/or hydroxypropyl methylcellulose (HPMC).

In at least one embodiment, the present invention provides a device for providing a controlled release of a pharmaceutically active ingredient contained therein, the device comprising at least one selected pharmaceutically active ingredient incorporated within a homogeneous matrix comprising effective amounts of two intelligent polymers having opposing wettability characteristics, wherein one polymer is selected which demonstrates a stronger tendency towards hydrophobicity and the other polymer is selected which demonstrates a stronger tendency towards hydrophilicity.

In at least one embodiment, the composition and device of the present invention can be provided as a tablet and may be optionally encased in a coating material. In at least one embodiment, the coating material prevents the burst and/or food effect associated with orally ingested medicaments and imparts gastrointestinal stealth characteristics. In at least one embodiment, the encoated matrix provides controlled release kinetics comparable to those of osmotic or press coated controlled release devices. In at least one embodiment, the composition is provided for oral administration or as a suppository depending on the chosen pharmaceutical active agent selected therein.

In at least one embodiment, the present invention provides a controlled release drug delivery system for the effective delivery of one or more of the following pharmaceutically active ingredients: topiramate, nifedipine, nicardipine, felodipine, captopril, naproxen, diclofenac, terfenadine, pentoxifylline, fenofibrate, glipizide, buspirone, cisapride, verapamil, diltiazem, aciclovir, zidovudine, pilocarpine, moclobemide, lamotrigine, risperidon, clonazepam, nefazodone, lovastatin, simvastatin, pravachol, ketorolac, hydromorphone, morphine, ticlopidine, seligiline, bupropion, venlafaxine, alprazolam, carbamazepine, divalproex and phenytoin.

In at least one embodiment, the present invention provides controlled delivery of therapeutic agents selected from the group consisting of anti-histamines, anti-depressants, anti-viral agents, anesthetics, antacids, anti-arthritics, antibiotics, anti-psychotics, anti-spasmodics, anxiolytic agents, appetite suppressants, cardiovascular agents, cough suppressants, emollients, gastro-intestinal agents, growth regulators, hypoglycemic agents, respiratory stimulants, vitamins, angiotensin converting enzyme inhibitors, anti-asthmatics, anti-cholesterolemics, anti-convulsants, anti-depressants, anti-diarrhea preparations, anti-infectives, anti-inflammatory agents, anti-nauseants, anti-stroke agents, anti-tumor drugs, anti-tussives, anti-uricemic drugs, amino-acid preparations, antiemetics, antiobesity drugs, antiparasitics, antipyretics, appetite stimulants, cerebral dilators, chelating agents, cholecystokinin antagonists, cognition activators, deodorants, dermatological agents, diabetes agents, diuretics, erythropoietic drugs, fertility agents, synthetic hormones, laxatives, mineral supplements, neuroleptics, neuromuscular agents, peripheral vaso-dilators, prostaglandins, vaginal preparations, vaso-constrictors and vertigo agents; acetaminophen, acetic acid, acetylsalicylic acid, buffered acetylsalicylic acid, albuterol, albuterol sulfate, ethanol, isopropanol, allantoin, aloe, aluminum acetate, aluminum carbonate, aluminum chlorohydrate, aluminum hydroxide, alprozolam, amino acids, aminobenzoic acid, amoxicillin, ampicillin, amsacrine, amsalog, anethole, aspartame, atenolol, bacitracin, balsam peru, beclomethasone dipropionate, benzocaine, benzoic acid, benzophenones, benzoylperoxide, biotin, bisacodyl, bornyl acetate, bromopheniramine maleate, bupropion, buspirone, caffeine, calamine, calcium, calcium carbonate, calcium casinate, calcium hydroxide, camphor, captopril, cascara sagrada, castor oil, cefaclor, cefadroxil, cephalexin, cetylalcohol, cetylpyridinium chloride, chelated minerals, chloramphenicol, chlorcyclizine hydrochloride, chlorhexidine gluconate, chloroxylenol, chloropentostatin, chlorpheniramine maleate, cholestyramine resin, choline bitartrate, cimetidine hydrochloride, cinnamedrine hydrochloride, citalopram, citric acid, cocoa butter, cod liver oil, codeine and codeine phosphate, clonidine, clonidine hydrochloride, clorfibrate, ciprofloxacin HCl, cyanocobalamin, cyclizine hydrochloride, danthron, dexbrompheniramine maleate, dextromethorphan hydrobromide, diazepam, dibucaine, diclofenac sodium, digoxin, dimethicone, dioxybenzone, diphenhydramine citrate, diphenhydramine hydrochloride, docusate calcium, docusate potassium, docusate sodium, doxycycline hyclate, doxylamine succinate, efaroxan, enalapril, enoxacin, erythromycin, estropipate, ethinyl estradiol, ephedrine, epinephrine bitartrate, erythropoietin, eucalyptol, ferrous fumarate, ferrous gluconate, ferrous sulfate, folic acid, fosphenytoin, fluoxetine HCl, furosemide, gabapentan, gentamicin, gemfibrozil, glipizide, glycerin, glyceryl stearate, griseofulvin, guaifenesin, hexylresorcinol, hydrochlorothiazide, hydrocodone bitartrate, hydrocortisone, hydrocortisone acetate, 8-hydroxyquinoline sulfate, ibuprofen, indomethacin, inositol, insulin, iodine, ipecac, iron, isoxicam, ketamine, kaolin, lactic acid, lanolin, lecithin, lidocaine, lidocaine hydrochloride, lifinopril, liotrix, lovastatin, magnesium carbonate, magnesium salicylate, magnesium trisilicate, mefenamic acid, meclofenamic acid, meclofenamate sodium, medroxyprogesterone acetate, methenamine mandelate, menthol, meperidine hydrochloride, metaproterenol sulfate, methyl nicotinate, methyl salicylate, methylcellulose, methsuximide, metromidazole, metromidazole hydrochloride, metoprolol tartrate, miconazole nitrate, mineral oil, minoxidil, morphine, naproxen sodium, neomycin sulfate, niacin, niacinamide, nicotine, nicotinamide, nitroglycerin, nonoxynol-9, norethindrone, norethindrone acetate, nystatin, octoxynol, octyl dimethyl PABA, octyl methoxycinnamate, omega-3 polyunsaturated fatty acids, omeprazole, oxolinic acid, oxybenzone, oxtriphylline, para-aminobenzoic acid (PABA), padimate, paramethadione, pentastatin, peppermint oil, pentaerythritol tetranitrate, pentobarbital sodium, pheniramine maleate, phenobarbital, phenol, phenolphthalein, phenylephrine hydrochloride, phenylpropanolamine, phenylpropanolamine hydrochloride, phenytoin, phenelzine sulfate, pirmenol, piroxicam, polymycin B sulfate, potassium chloride, potassium nitrate, prazepam, procainamide hydrochloride, procaterol, propoxyphene, propoxyphene HCl, propoxyphene napsylate, pramiracitin, pramoxine, pramoxine hydrochloride, propranolol HCl, pseudoephedrine hydrochloride, pseudoephedrine sulfate, pyridoxine, quinapril, quinidine gluconate, quinestrol, ralitoline, ranitadine, resorcinol, riboflavin, salicylic acid, sesame oil, shark liver oil, simethicone, sodium bicarbonate, sodium citrate, sodium fluoride, sodium monofluorophosphate, sulfanethoxazole, sulfur, tacrine, tacrine HCl, theophylline, tramadol, terfenidine, thioperidone, trimetrexate, triazolam, timolol maleate, tretinoin, tetracycline hydrochloride, tolmetin, tolnaftate, triclosan, triprolidine hydrochloride, topiramate, undecylenic acid, vancomycin, vidaribine phosphate, vitamin A, vitamin B, vitamin C, vitamin D, vitamin E, vitamin K, witch hazel, xylometazoline hydrochloride, zinc, zinc sulfate, and zincundecylenate.

In accordance with another aspect of the present invention is a method for preparing a device for the controlled release of selected pharmaceutically active ingredients, the method comprising blending at least one selected pharmaceutically active substance with about 5 to 25% by weight hydrophillic polymer and about 1 to 25% hydrophobic polymer, adding suitable pharmaceutical excipients, surface active agents and lubricants, granulating the mixture with isopropyl alcohol, drying the granular mixture, milling the dried mixture, adding about 5 to 70% ethylcellulose, adding a lubricant and optionally a glidant and compressing the granules into tablets. The tablets are optionally encased in a gastrointestinal stealth encasement or a pharmaceutically acceptable film coat.

### BRIEF DESCRIPTION OF THE DRAWINGS

FIG. 1 is a graph showing the dissolution profile at pH 6.8 of topiramate 200 mg tablets prepared according to Example 10.

FIG. 2 is a graph showing the dissolution profile at pH 4.5 of topiramate 200 mg tablets prepared according to Example 10.

FIG. 3 is a graph comparing the mean plasma topiramate concentration-time profiles under fasting conditions for topiramate 200 mg tablets, Formulations A and B, prepared according to Example 10, administered q.d. and for Topamax® 100 mg tablets administered b.i.d.

FIG. 4 is a graph comparing the mean plasma topiramate concentration-time profiles under fasting conditions for topiramate 200 mg tablets, Formulations C and D, prepared according to Example 10, administered q.d. and for Topamax® 100 mg tablets administered b.i.d.

FIG. 5 is a graph comparing predicted plasma topiramate concentration-time profiles from steady state simulation of repeated administration for 14 days of topiramate 200 mg tablets, Formulations A and B, prepared according to Example 10, administered q.d. and Topamax® 100 mg tablets administered b.i.d.

FIG. 6 is a graph comparing predicted plasma topiramate concentration-time profiles from steady state simulation of repeated administration for 14 days of topiramate 200 mg tablets, Formulations C and D, prepared according to Example 10, administered q.d. and Topamax® 100 mg tablets administered b.i.d.

FIG. 7 is a graph comparing the deconvoluted plasma topiramate concentration-time profiles to the dissolution profiles at pH 6.8 of topiramate 200 mg tablets, Formulations A, B and C, prepared according to Example 10, administered q.d.

FIG. 8 is a graph showing the in vitro/in vivo correlation for topiramate 200 mg tablets prepared according to Example 10, based on dissolution data obtained at pH 6.8.

FIG. 9 is a graph comparing the actual plasma topiramate concentration-time profile obtained for topiramate 200 mg tablets, Formulation A, prepared according to Example 10, administered q.d., to the predicted plasma topiramate concentration-time profile based on in vitro/in vivo correlation.

FIG. 10 is a graph comparing the actual plasma topiramate concentration-time profile obtained for topiramate 200 mg tablets, Formulation B, prepared according to Example 10, administered q.d., to the predicted plasma topiramate concentration-time profile based on in vitro/in vivo correlation.

FIG. 11 is a graph comparing the actual plasma topiramate concentration-time profile obtained for topiramate 200 mg tablets, Formulation C, prepared according to Example 10, administered q.d., to the predicted plasma topiramate concentration-time profile based on in vitro/in vivo correlation.

### DETAILED DESCRIPTION OF THE INVENTION

The present invention provides a composition which provides controlled sustained release of pharmaceutically active ingredients. In at least one embodiment, the composition demonstrates stealth characteristics. In at least one embodiment, the composition of the present invention provides a pseudo first order, first order or zero order release of pharmaceutically active substances.

In at least one embodiment, the composition is a matrix tablet. As used herein interchangeably, the terms “matrix tablet” or “controlled release matrix tablet” are intended to mean a unitary tablet containing a core in which the pharmaceutically active ingredient is dispersed homogeneously within a matrix which acts to provide controlled release of the pharmaceutically active ingredient. The release rate of the pharmaceutically active ingredient from the core can be modified by the addition of pore-forming hydrophilic salts, flux or channeling agents, hydroattractants, solutes, wicking agents, or wetting aids, or by manipulation of processing parameters such as the compression force or the particle size of the materials used in the preparation of the matrix tablet. In at least one embodiment, the matrix tablet is uncoated. In at least one embodiment, the matrix tablet is coated with at least one functional or non-functional coat.

In at least one embodiment, the composition is a matrix tablet in which the pharmaceutically active ingredients are intimately mixed with two groups of intelligent polymers having opposing wettability characteristics, the first intelligent polymer component demonstrating a stronger tendency towards hydrophobicity, for example, ethylcellulose (EC), and the second intelligent polymer component possessing a stronger tendency towards hydrophilicity, for example, hydroxyethylcellulose (HEC) or hydroxypropylmethylcellulose (HPMC). In at least one embodiment, the amount of ethylcellulose in the composition is not less than about 5% wt/wt, and preferably, is about 5% to about 70% wt/wt of the final formulation. In at least one embodiment, the second intelligent polymer component is present in the composition in an amount of from about 15% to about 50% by weight. In at least one embodiment, the HEC and HPMC are present in a ratio of about 1:100 to about 100:1, the preferred ratio being from about 1:50 to about 50:1. In at least one embodiment, the intelligent polymers together provide a homogeneous matrix for the pharmaceutically active ingredient and have the following single and three-component calculated solubility parameters (MPa^{0.5}) using the group contribution method.

| Wettability of | | | | | | |
|---|---|---|---|---|---|---|
| polymer | δ | δₜ | δ_{d} | δₚ | δₕ | δ₋ₐ |
| More hydrophilic | 18-50 | 18-45 | 12-17 | 2-8 | 12-20 | 13-20 |
| intelligent polymer | | | | | | |
| More hydrophobic | 15-25 | 14-24 | 12-17 | 2-7 | 5-15 | 6-13 |
| intelligent polymer | | | | | | |

where δ is the conventional Hildebrand parameter, ₜ= total, _{d}= dispersion, ₚ= polar, ₕ= hydrogen bond and ₐ= association interactions.

In at least one embodiment, the composition comprises at least one suitable pharmaceutically acceptable excipient. Excipients which may be used in the compositions of the present invention include but are not limited to glidants, surface active agents, channeling agents, lubricants and compression enhancers. Although examples of suitable excipients, glidants, surface active agents, channeling agents, lubricants, and compression enhancers are listed herein, it is understood by those skilled in the art that other suitable excipients, glidants, surface active agents, channeling agents, lubricants, and compression enhancers may also be used in the present invention. One skilled in the art would clearly be able to identify the excipients, glidants, surface active agents, channeling agents, lubricants, and compression enhancers suitable for use in the present invention.

Suitable glidants are optionally present in an amount of about 0.25% to about 5% wt/wt and include but are not limited to talc, silicon dioxide, silica aerogels (Cab-O-Sils, Syloids), calcium silicate, magnesium stearate, zinc stearate, starch, magnesium lauryl sulfate, sodium lauryl sulfate, magnesium oxide and magnesium carbonate. In at least one embodiment, the glidant is silicon dioxide.

Suitable surface active agents are optionally present in the amount of up to about 15% wt/wt and include but are not limited to fatty alcohols; nonionic esters including but not limited to ethylene glycol esters, propylene glycol esters, glyceryl esters, polyglyceryl esters, sorbitan esters, sucrose esters and ethoxylated esters; nonionic ethers including but not limited to fatty alcohol ethoxylates, propoxylated alcohols and ethoxylated/propoxylated block polymers; alkylpolyglycosides; alkanolamides; carboxylates including but not limited to acyl lactylates, ether carboxylates, acyl amides of anion acids and natural emulsifiers; esters of sulfuric acids including alkyl sulfates and ethoxylated alkyl sulfates; sulfonates including but not limited to linear alkyl benzene sulfonates, α-olefin sulfonates, alkyl glyceryl ether sulfonates, alkylether sufonates, acyl isethionates, acyl taurates and sulfosuccinates; phosphoric acid esters; quarternary ammonium salts; betaines; ethoxylated amines; acrylic acid derivatives; substituted alkylamides; phosphatides; amine oxides; perfluorinated alkyl derivatives, starch-derived surfactants, polymeric surfactants, beeswax, lanolin and ethoxylated polysiloxane. In at least one embodiment, suitable surface active agents include but are not limited to sodium lauryl sulfate and block copolymers of polyoxyethylene and polyoxypropylene, including but not limited to poloxamers such as, for example, poloxamer F127, poloxamer 407 or Pluronic® F127. It has been unexpectedly found that when the composition comprises topiramate or a pharmaceutically acceptable salt thereof, the presence of a surface active agent is optional. Therefore, in at least one embodiment, the composition comprises a surface active agent. Furthermore, in at least one embodiment, the composition is free of a surface active agent.

Channeling agents may be present in an amount of about 10% to about 70% wt/wt and include but are not limited to lactose, sucrose, sorbitol, mannitol, sodium chloride and potassium chloride. In at least one embodiment, the channeling agent is lactose (including but not limited to anhydrous lactose, lactose monohydrate and spray dried lactose).

Suitable lubricants for use in the composition are present in an amount of about 0.1% to about 5% and include but are not limited to magnesium stearate, calcium stearate, zinc stearate, talc, starch, glyceryl behenate, sodium stearyl fumarate, light mineral oil, stearic acid, hydrogenated vegetable oils, polyethylene glycols, sodium acetate, sodium benzoate, sodium chloride, leucine, sodium lauryl sulfate and magnesium lauryl sulfate. In at least one embodiment, the lubricant is magnesium stearate.

Compression enhancers may be present in an amount of about 5% to about 30% wt/wt and include but are not limited to microcrystalline cellulose, dextrose, maltodextrins, sorbitol, mannitol, dicalcium phosphate and modified starches. In at least one embodiment, the compression enhancer is microcrystalline cellulose.

In at least one embodiment, the pharmaceutically active ingredients are selected from those substances that have a water contact angle (θ) such that cos θ is between +0.9848 and −0.9848. The composition may contain one or more such active ingredients, or a pharmaceutically acceptable salt thereof, in an amount to provide therapeutically effective dosages. In at least one embodiment, the pharmaceutically active ingredients may be selected from, but are not limited to, topiramate, nifedipine, nicardipine, felodipine, captopril, naproxen, diclofenac, terfenadine, pentoxifylline, fenofibrate, glipizide, buspirone, cisapride, verapamil, diltiazem, aciclovir, zidovudine, pilocarpine, moclobemide, lamotrigine, risperidon, clonazepam, nefazodone, lovastatin, simvastatin, pravachol, ketorolac, hydromorphone, morphine, ticlopidine, seligiline, bupropion, venlafaxine, alprazolam, carbamazepine, divalproex and phenytoin.

Furthermore, in at least one embodiment, therapeutic agents which may also be used in the composition of the present invention are selected from the group consisting of anti-histamines, anti-depressants, anti-viral agents, anesthetics, antacids, anti-arthritics, antibiotics, anti-psychotics, anti-spasmodics, anxiolytic agents, appetite suppressants, cardiovascular agents, cough suppressants, emollients, gastro-intestinal agents, growth regulators, hypoglycemic agents, respiratory stimulants, vitamins, angiotensin converting enzyme inhibitors, anti-asthmatics, anti-cholesterolemics, anti-convulsants, anti-depressants, anti-diarrhea preparations, anti-infectives, anti-inflammatory agents, anti-nauseants, anti-stroke agents, anti-tumor drugs, anti-tussives, anti-uricemic drugs, amino-acid preparations, antiemetics, antiobesity drugs, antiparasitics, antipyretics, appetite stimulants, cerebral dilators, chelating agents, cholecystokinin antagonists, cognition activators, deodorants, dermatological agents, diabetes agents, diuretics, erythropoietic drugs, fertility agents, synthetic hormones, laxatives, mineral supplements, neuroleptics, neuromuscular agents, peripheral vaso-dilators, prostaglandins, vaginal preparations, vaso-constrictors and vertigo agents; acetaminophen, acetic acid, acetylsalicylic acid, buffered acetylsalicylic acid, albuterol, albuterol sulfate, ethanol, isopropanol, allantoin, aloe, aluminum acetate, aluminum carbonate, aluminum chlorohydrate, aluminum hydroxide, alprozolam, amino acids, aminobenzoic acid, amoxicillin, ampicillin, amsacrine, amsalog, anethole, aspartame, atenolol, bacitracin, balsam peru, beclomethasone dipropionate, benzocaine, benzoic acid, benzophenones, benzoylperoxide, biotin, bisacodyl, bornyl acetate, bromopheniramine maleate, bupropion, buspirone, caffeine, calamine, calcium, calcium carbonate, calcium casinate, calcium hydroxide, camphor, captopril, cascara sagrada, castor oil, cefaclor, cefadroxil, cephalexin, cetylalcohol, cetylpyridinium chloride, chelated minerals, chloramphenicol, chlorcyclizine hydrochloride, chlorhexidine gluconate, chloroxylenol, chloropentostatin, chlorpheniramine maleate, cholestyramine resin, choline bitartrate, cimetidine hydrochloride, cinnamedrine hydrochloride, citalopram, citric acid, cocoa butter, cod liver oil, codeine and codeine phosphate, clonidine, clonidine hydrochloride, clorfibrate, ciprofloxacin HCl, cyanocobalamin, cyclizine hydrochloride, danthron, dexbrompheniramine maleate, dextromethorphan hydrobromide, diazepam, dibucaine, diclofenac sodium, digoxin, dimethicone, dioxybenzone, diphenhydramine citrate, diphenhydramine hydrochloride, docusate calcium, docusate potassium, docusate sodium, doxycycline hyclate, doxylamine succinate, efaroxan, enalapril, enoxacin, erythromycin, estropipate, ethinyl estradiol, ephedrine, epinephrine bitartrate, erythropoietin, eucalyptol, ferrous fumarate, ferrous gluconate, ferrous sulfate, folic acid, fosphenytoin, fluoxetine HCl, furosemide, gabapentan, gentamicin, gemfibrozil, glipizide, glycerin, glyceryl stearate, griseofulvin, guaifenesin, hexylresorcinol, hydrochlorothiazide, hydrocodone bitartrate, hydrocortisone, hydrocortisone acetate, 8-hydroxyquinoline sulfate, ibuprofen, indomethacin, inositol, insulin, iodine, ipecac, iron, isoxicam, ketamine, kaolin, lactic acid, lanolin, lecithin, lidocaine, lidocaine hydrochloride, lifinopril, liotrix, lovastatin, magnesium carbonate, magnesium salicylate, magnesium trisilicate, mefenamic acid, meclofenamic acid, meclofenamate sodium, medroxyprogesterone acetate, methenamine mandelate, menthol, meperidine hydrochloride, metaproterenol sulfate, methyl nicotinate, methyl salicylate, methylcellulose, methsuximide, metromidazole, metromidazole hydrochloride, metoprolol tartrate, miconazole nitrate, mineral oil, minoxidil, morphine, naproxen sodium, neomycin sulfate, niacin, niacinamide, nicotine, nicotinamide, nitroglycerin, nonoxynol-9, norethindrone, norethindrone acetate, nystatin, octoxynol, octyl dimethyl PABA, octyl methoxycinnamate, omega-3 polyunsaturated fatty acids, omeprazole, oxolinic acid, oxybenzone, oxtriphylline, para-aminobenzoic acid (PABA), padimate, paramethadione, pentastatin, peppermint oil, pentaerythritol tetranitrate, pentobarbital sodium, pheniramine maleate, phenobarbital, phenol, phenolphthalein, phenylephrine hydrochloride, phenylpropanolamine, phenylpropanolamine hydrochloride, phenytoin, pheneizine sulfate, pirmenol, piroxicam, polymycin B sulfate, potassium chloride, potassium nitrate, prazepam, procainamide hydrochloride, procaterol, propoxyphene, propoxyphene HCl, propoxyphene napsylate, pramiracitin, pramoxine, pramoxine hydrochloride, propranolol HCl, pseudoephedrine hydrochloride, pseudoephedrine sulfate, pyridoxine, quinapril, quinidine gluconate, quinestrol, ralitoline, ranitadine, resorcinol, riboflavin, salicylic acid, sesame oil, shark liver oil, simethicone, sodium bicarbonate, sodium citrate, sodium fluoride, sodium monofluorophosphate, sulfanethoxazole, sulfur, tacrine, tacrine HCl, theophylline, tramadol, terfenidine, thioperidone, trimetrexate, triazolam, timolol maleate, tretinoin, tetracycline hydrochloride, tolmetin, tolnaftate, triclosan, triprolidine hydrochloride, topiramate, undecylenic acid, vancomycin, vidaribine phosphate, vitamin A, vitamin B, vitamin C, vitamin D, vitamin E, vitamin K, witch hazel, xylometazoline hydrochloride, zinc, zinc sulfate, and zincundecylenate.

In at least one embodiment, the pharmaceutically active ingredient is nifedipine which provides coronary vasodilating and hypotensive effects. As this medicament is hardly water soluble, has little absorbability in body fluids and is rapidly metabolized and excreted, it is highly advantageous to provide nifedipine in the controlled release composition of the present invention. In at least one embodiment, the pharmaceutically active ingredient is topiramate which has anticonvulsant activity. In at least one embodiment, the pharmaceutically active ingredient is selected from glipizide, diltiazem hydrochloride, verapamil hydrochloride, buspirone hydrochloride, tramadol hydrochloride, bupropion hydrobromide and bupropion hydrochloride.

The terms “pharmaceutically active ingredient” or “therapeutic agent” used herein interchangeably refer to substances which provide a therapeutic effect and include, but are not limited to, pharmaceutically acceptable salts of such substances.

The term “pharmaceutically acceptable salt” as used herein is intended to mean a salt of the pharmaceutically active ingredient which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically acceptable acid addition salts and pharmaceutically acceptable base addition salts. Lists of suitable salts are found in, for example, S. M. Berge et al., J. Pharm. Sci. (1977) 66(1):1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, hydroiodic acid, sulfuric acid, sulfurous acid, sulfamic acid, nitric acid, phosphoric acid, carbonic acid and the like, and organic acids including but not limited to acetic acid, acrylic acid, trifluoroacetic acid, adipic acid, ascorbic acid, aspartic acid, benzenesulfonic acid, benzoic acid, p-bromobenzenesulfonic acid, butynedioic acid, butyric acid, camphoric acid, camphorsulfonic acid, caproic acid, caprylic acid, chlorobenzoic acid, cinnamic acid, citric acid, decanoic acid, digluconic acid, dinitrobenzoic acid, ethanesulfonic acid, formic acid, fumaric acid, gluconic acid, glutamic acid, glycolic acid, glycerophosphoric acid, hemisulfic acid, hexanoic acid, hexynedioic acid, heptanoic acid, hydroxybenzoic acid, gamma-hydroxybutyric acid, 2-hydroxyethanesulfonic acid (isethionic acid), hydroxymaleic acid, isobutyric acid, lactic acid, maleic acid, malic acid, malonic acid, mandelic acid, mesitylenesulfonic acid, methanesulfonic acid, methoxybenzoic acid, methylbenzoic acid, mucic acid, naphthalenesulfonic acid, nicotinic acid, 2-naphthalenesulfonic acid, oxalic acid, pamoic acid, pantothenic acid, pectinic acid, phenylacetic acid, phenylbutyric acid, 3-phenylpropionic acid, phthalic acid, pivalic acid, propanesulfonic acid, propiolic acid, propionic acid, pyruvic acid, salicylic acid, sebacic acid, stearic acid, suberic acid, succinic acid, sulfanilic acid, tartaric acid, p-toluenesulfonic acid, undecanoic acid, xylenesulfonic acid, and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like, and pharmaceutically-acceptable organic nontoxic bases including but not limited to primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, propylamine, butylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like.

In at least one embodiment, the composition of the present invention comprises topiramate or a pharmaceutically acceptable salt thereof and exhibits bioequivalence to a reference formulation of topiramate or a pharmaceutically acceptable salt thereof. As used herein, a “reference formulation” is intended to mean a formulation of topiramate or a pharmaceutically acceptable salt thereof which is currently approved for marketing and which may be used as a reference for a new drug application (NDA) or an abbreviated new drug application (ANDA) under the Federal Food Drug & Cosmetic Act. Currently marketed formulations of topiramate or a pharmaceutically acceptable salt thereof include Topamax® (currently approved in the US under NDA #020505) and Topamax Sprinkle® (currently approved in the US under NDA #020844). As used herein, the terms “bioequivalent” or “bioequivalence” are intended to mean bioequivalent or bioequivalence according to current US Food and Drug Administration (FDA) guidelines. According to current US FDA guidelines (Guidance for Industry: Statistical Approaches to Establishing Bioequivalence, last revised January 2001; and Guidance for Industry: Bioavailability and Bioequivalence Studies for Orally Administered Drug Products-General Considerations, last revised March 2003), in order for a test drug product to show bioequivalence to a reference formulation (the reference drug product), the 90% confidence intervals determined for the ratio of the averages (population geometric means) of the values of Cₘₐₓ and AUC for the test and reference drug products must fall within the limits of 80.00% to 125.00%.

In at least one embodiment, the composition of the present invention comprises topiramate or a pharmaceutically acceptable salt thereof and exhibits a dissolution profile at pH 4.5 or at pH 6.8 characterized by the following equation:

y=100−100*e^{(-a*x}^{^{b}})

where:

y=% dissolution;

x=sampling time;

a=scale parameter which ranges from about 0.06 to about 0.12;

b=shape parameter which ranges from about 0.9 to about 1.6; and

100=the cumulative percentage of the topiramate released at time infinity.

This equation describes a mathematical function well known in the art as a Weibull distribution (Polli, J. E. et al, Drug Information Journal (1996) 30:1113-20; Costa, P. and J. M. S. Lobo, European Journal of Pharmaceutical Sciences (2001) 13:123-33; Langenbucher, F., J. Pharm. Pharmacol. (1972) 24:979).

In at least one embodiment, the composition of the present invention comprises topiramate or a pharmaceutically acceptable salt thereof and exhibits a dissolution profile at pH 6.8 characterized by the following equation:

y=100−100*e^{(-a*x}^{^{b}})
where:

y=% dissolution;

x=sampling time;

a=scale parameter which ranges from about 0.06 to about 0.12;

b=shape parameter which ranges from about 0.9 to about 1.5; and

100=the cumulative percentage of the topiramate released at time infinity.

In at least one embodiment, the composition of the present invention comprises topiramate or a pharmaceutically acceptable salt thereof and exhibits a dissolution profile at pH 4.5 characterized by the following equation:

y=100−100*e^{(-a*x}^{^{b}})

where:

y=% dissolution;

x=sampling time;

a=scale parameter which ranges from about 0.06 to about 0.12;

b=shape parameter which ranges from about 1.1 to about 1.6; and

100=the cumulative percentage of the topiramate released at time infinity.

In at least one embodiment, the controlled release matrix tablet comprising topiramate or a pharmaceutically acceptable salt thereof exhibits an in vitro dissolution profile at pH 6.8 or 4.5 such that after about 1 hour, no more than about 15% of the topiramate or pharmaceutically acceptable salt thereof is released; after about 2 hours, from about 5% to about 35% of the topiramate or pharmaceutically acceptable salt thereof is released; after about 4 hours, from about 20% to about 60% of the topiramate or pharmaceutically acceptable salt thereof is released; after about 8 hours, from about 35% to about 95% of the topiramate or pharmaceutically acceptable salt thereof is released; after about 12 hours, no less than about 50% of the topiramate or pharmaceutically acceptable salt thereof is released; and after about 16 hours, no less than about 60% of the topiramate or pharmaceutically acceptable salt thereof is released.

In at least one embodiment, the controlled release matrix tablet comprising topiramate or a pharmaceutically acceptable salt thereof exhibits a dissolution profile at pH 6.8 such that after about 1 hour, no more than about 15% of the topiramate or pharmaceutically acceptable salt thereof is released; after about 2 hours, from about 15% to about 35% of the topiramate or pharmaceutically acceptable salt thereof is released; after about 4 hours, from about 30% to about 60% of the topiramate or pharmaceutically acceptable salt thereof is released; after about 8 hours, from about 55% to about 90% of the topiramate or pharmaceutically acceptable salt thereof is released; after about 12 hours, no less than about 75% of the topiramate or pharmaceutically acceptable salt thereof is released; and after about 16 hours, no less than about 85% of the topiramate or pharmaceutically acceptable salt thereof is released.

In at least one embodiment, the controlled release matrix tablet comprising topiramate or a pharmaceutically acceptable salt thereof exhibits a dissolution profile at pH 4.5 such that after about 1 hour, no more than about 15% of the topiramate or pharmaceutically acceptable salt thereof is released; after about 2 hours, from about 20% to about 30% of the topiramate or pharmaceutically acceptable salt thereof is released; after about 4 hours, from about 40% to about 60% of the topiramate or pharmaceutically acceptable salt thereof is released; after about 8 hours, from about 65% to about 95% of the topiramate or pharmaceutically acceptable salt thereof is released; and after about 12 hours, no less than about 90% of the topiramate or pharmaceutically acceptable salt thereof is released.

Surprisingly, in at least one embodiment, the controlled release matrix tablet comprising topiramate or a pharmaceutically acceptable salt thereof exhibits an in vitro/in vivo correlation (IVIVC). According to current US FDA guidelines (Guidance for Industry: Extended Release Oral Dosage Forms: Development, Evaluation and Application of In Vitro/In Vivo Correlations, last revised September 1997), an in vitro/in vivo correlation is a predictive mathematical model describing the relationship between an in vitro property of an extended release dosage form (usually the rate or extent of drug dissolution or release) and a relevant in vivo response, e.g., plasma drug concentration or amount of drug absorbed. As is well known in the art, such a correlation allows for the prediction of in vivo concentration or absorption profiles from dissolution profiles determined in vitro.

The composition can be uncoated or can comprise at least one functional or non-functional coat. The term “coat” as used herein is defined to mean a coating substantially surrounding a core which provides desirable properties to the dosage form. As is clear to the person of skill in the art, the coat can serve several purposes, including but not limited to protecting the dosage form from environmental conditions, such as light or moisture, providing esthetic or taste-masking properties to the dosage form, making the dosage form easier to swallow or to handle during the production process, or modifying the release properties of the dosage form, such that pharmaceutically active ingredient is released at a different rate from the coated core than from the uncoated core. A coat can itself comprise one or more pharmaceutically active ingredients. One or more than one coat, with the same or different functions or properties, can be applied to a core. The term “coat” includes, but is not limited to, modified release coats, immediate release coats and non-functional soluble coats.

The term “modified release coat” as used herein is defined to mean a coat which when applied onto an uncoated core will desirably provide modified release of the pharmaceutically active ingredient from the core, compared to the rate of release from the uncoated core. Modified release coats include but are not limited to controlled release coats and delayed release coats.

The term “controlled release coat” as used herein is defined to mean a coat which when applied onto an uncoated core will desirably slow the rate of release of the pharmaceutically active ingredient from the core, compared to the rate of release from the uncoated core. Such a coat can, for example, comprise at least one pH independent polymer; at least one pH dependent polymer, including but not limited to enteric or reverse enteric types; at least one soluble material, including but not limited to a soluble polymer; at least one insoluble material, including but not limited to an insoluble polymer; at least one swellable material, including but not limited to a swellable polymer; at least one swellable and erodable material, including but not limited to a swellable and erodable polymer; at least one hydrophobic material, or combinations thereof. The controlled release coat will desirably be designed such that when the coat is applied to a core, the dosage form in conjunction with the controlled release coat will desirably exhibit controlled release of the pharmaceutically active ingredient. The “controlled release coat” can optionally comprise additional materials that may alter the functionality of the controlled release coat.

A “delayed release coat” as used herein is defined to mean a functional coat which, when applied onto a core, does not allow appreciable drug release immediately following administration but at a later time. Delayed release coats provide a time delay prior to the commencement of drug release. Such a coat desirably comprises at least one pH dependent polymer, including but not limited to enteric or reverse enteric polymers, but can, in addition, comprise other hydrophilic or hydrophobic polymers and other pharmaceutically acceptable excipients to either facilitate processing of the delayed release coat or to alter the functionality of the coat. Other components of the delayed release coat include but are not limited to pH independent polymers; soluble materials, including but not limited to soluble polymers; insoluble materials, including but not limited to insoluble polymers; swellable materials, including but not limited to swellable polymers; lipids; waxy materials; hydrophobic materials; hydrophilic materials; or combinations thereof. A delayed release coat will desirably be applied onto a core such that after administration, the coat, either by dissolving slowly or disruption under certain pH conditions, allows release from the core to begin not in the stomach but in some predetermined region of the small intestine or even further down the intestinal tract, such as for example, in the colon. Coats comprising enteric materials, including but not limited to enteric polymers, will fall under the definition of a delayed release coat. A delayed release coat can be applied to a modified release core so as to delay the release of the pharmaceutically active ingredient followed by a modified release of the pharmaceutically active ingredient.

An “immediate release” coat or a “non-functional soluble coat”, as used herein interchangeably, is defined to mean a coat which has substantially no influence on the rate of release of the pharmaceutically active ingredient from the dosage form in-vitro or in-vivo. The excipients comprising the immediate release coat have no substantial modified release properties. Such a coat can function to provide immediate release of an pharmaceutically active ingredient from a dosage form, and/or to enhance the chemical, biological or physical stability characteristics, or the physical appearance, of the dosage form.

Not to be bound by any theory, it is believed that the release of the pharmaceutically active ingredient within the present composition is provided due to the unique mixture of the rate controlling constituents and excipients in the selected ratios. When the composition is used as a matrix tablet, the solid pharmaceutically active ingredient dissolves from the outer surface of the matrix tablet first. When this surface becomes exhausted of pharmaceutically active ingredient, the underlying material begins to be depleted by dissolution and diffusion through the matrix to the external solution. During dissolution, some of the rate controlling constituents (the polymer blend) have a tendency towards swelling and thus act as a focus for cleavage or erosion of the matrix tablet. This leads to a cleavage of discrete amounts of pharmaceutically active ingredient in combination with the excipients in the composition at the point of contact or interface between the rate controlling constituents and the other ingredients. In this manner, the interface between the region containing dissolved pharmaceutically active ingredient and that containing dispersed pharmaceutically active ingredient recedes into the interior as a front. As the cleavage occurs, the pharmaceutically active ingredient is readily absorbed. The release rate becomes smaller towards the end of dissolution due to a reduction in volume of the tablet.

When the composition is coated with a delayed release coat, the release of pharmaceutically active ingredient can be influenced by the encasement coat surrounding the homogeneous matrix tablet in addition to the unique mixture of the rate controlling constituents and excipients in carefully selected ratios within the matrix tablet. Stepwise ionization of the surface groups of the coat triggered by the pH of the surrounding media and the resulting gradual dissolution of the coat over time exposes the matrix tablet to the fluids of the GI system. The solid pharmaceutically active ingredient when in contact with the fluids of the GI system dissolves from the outer surface of the matrix tablet first. When this surface becomes exhausted of pharmaceutically active ingredient the underlying material begins to be depleted by dissolution and diffusion through the matrix to the external solution.

In at least one embodiment, the present composition provides controlled release of pharmaceutically active ingredient over an extended period of time (up to at least 20 hours) with minimal initial dumping effects, such that the active ingredient is still being released from the composition 20 hours later.

In at least one embodiment, the compositions of the invention may be formulated in a tablet form or as a suppository. For each formulation, one or more coating compositions can be optionally applied. In at least one embodiment, the coating composition comprises anionic copolymers based on methacrylic acid and methyl methacrylate which are provided in an amount sufficient to obtain 0.5 to 15 mg per cm² on the tablet or suppository. This encasement coat, 0.5%-15% wt/wt, acts to minimize the initial burst effect seen in administered tableted compositions and also imparts gastrointestinal tract (GIT) “stealth” characteristics especially in the presence of food.

The present invention also provides a method for the manufacture of the novel controlled release pharmaceutical compositions in which the order and rate of drug release is dependant on the physicochemical properties and proportion of polymer blend and the wettability of the pharmaceutically active substance(s) such that sustained release effects are obtained therapeutically.

In at least one embodiment, a two step granulation technique is used to prepare a desired controlled release device containing at least one selected active ingredient. The method comprises intragranulation by wet granulation and extragranulation by dry granulation. In the intragranulation process the pharmaceutically active substance is blended with about 5-25% hydroxypropyl methylcellulose (preferably METHOCEL(R) premium grade type K4M PREM), about 1-20% hydroxyethylcellulose (preferably NATROSOL(R) 25OHHX), together with suitable pharmaceutical excipients including but not limited to glidants e.g., silicon dioxide (about 0.25-5%), surface active agents e.g., sodium lauryl sulfate (about 0.5-15%), channelling agents such as lactose (about 10-70%) and compression enhancers e.g., microcrystalline cellulose, AVICEL(R) 101 (about 5-30%), until a homogeneous mixture is obtained. Blending can be done in a V-blender but preferably in a planetary or high shear mixer. The homogeneous blend is then granulated with isopropyl alcohol (99%) in a planetary or high shear mixer. It is preferred that the granulating solvent is a non aqueous solvent.

The wet granules are dried in a fluid bed or in tray dryers to a loss on drying of <3% and organic volatile impurities of isopropyl alcohol about <15000 ppm. The dry granules are milled to about <1500 microns using a cone mill. Thereafter the extragranular addition of 5-70% of ethylcellulose having 30-60% ethoxyl content and vicosity 60-100 cps (preferably ETHOCEL(TM) type N100) to the dry milled granules is undertaken in a V-blender until a homogeneous blend is obtained. To this blend may be added a glidant, preferably talc, and a lubricant, preferably magnesium stearate. This final mixture is intimately blended and compressed into a matrix tablet using a rotary tablet press.

The matrix tablet can be used uncoated if no stealth characteristics are required. Under certain circumstances and for certain drugs, GIT stealth characteristics are desirable, e.g., in situations where dose dumping, burst or food effects are to be avoided. Stealth characteristics can be obtained by encasing the matrix tablet in a special coat composition consisting of anionic copolymer(s) based on methacrylic acid and methyl methacrylate. The preferred copolymers are the type A and/or Type B. This special composition may contain one or more of the following, plasticiser (about 0-25%), pigment (about 0-25%), glidant (about 0-30%), lubricant (about 0-30%). The values of dry polymer(s) encasing the matrix tablet in mg per cm² of surface area of tablet is about 0.5-15 mg per cm² . This special stealth encasement may be applied using a fluid bed or a conventional coating pan. It is preferable to use a side vented perforated coating pan in order to obtain a more uniform and efficient encasement. In at least one embodiment, the coating composition is aqueous based. In at least one embodiment, the coating composition is solvent based.

### EXAMPLES

The examples are described for the purposes of illustration and are not intended to limit the scope of the invention.

Methods of synthetic chemistry and pharmacology referred to but not explicitly described in this disclosure and examples are reported in the scientific literature and are well known to those skilled in the art.

### Example 1

### Glipizide ER 5 mg

| | % composition |
|---|---|
| Glipizide | 1.83 |
| Hydroxypropyl methylcellulose | 20 |
| Ethylcellulose | 16.17 |
| Hydroxyethylcellulose | 4 |
| Lactose | 30 |
| Microcrystalline cellulose | 23 |
| Silicone dioxide | 0.6 |
| Sodium Lauryl sulfate | 4 |
| Magnesium stearate | 0.4 |

### Example 2

### Diltiazem Hydrochloride ER 60 mg

| | % composition |
|---|---|
| Diltiazem hydrochloride | 58.82 |
| Hydroxypropyl methylcellulose | 5 |
| Ethylcellulose | 5 |
| Hydroxyethylcellulose | 15 |
| Lactose | 5 |
| Microcrystalline cellulose | 9.18 |
| Talc | 1 |
| Magnesium stearate | 1 |

### Example 3

### Nifedipine ER 60 mg

| | % composition |
|---|---|
| Nifedipine | 20 |
| Hydroxypropyl methylcellulose | 20 |
| Ethylcellulose | 29 |
| Hydroxyethylcellulose | 3.8 |
| Lactose | 14 |
| Microcrystalline cellulose | 10 |
| Silicone dioxide | 1.2 |
| Na lauryl sulfate | 1 |
| Magnesium stearate | 1 |

### Example 4

### Verapamil Hydrochloride ER 60 mg

| | % composition |
|---|---|
| Verapamil HCl | 50 |
| Hydroxypropyl methylcellulose | 10 |
| Ethylcellulose | 5 |
| Hydroxyethylcellulose | 8 |
| Lactose | 16 |
| Microcrystalline cellulose | 10 |
| Magnesium stearate | 1 |

### Example 5

### Diltiazem Hydrochloride/Hydrochlorothiazide ER 60/12.5 mg

| | % composition |
|---|---|
| Diltiazem hydrochloride | 48 |
| Hydrochlorothiazide | 10 |
| Hydroxypropyl methylcellulose | 5.82 |
| Ethylcellulose | 5 |
| Hydroxyethylcellulose | 15 |
| Lactose | 5 |
| Microcrystalline cellulose | 9.18 |
| Talc | 1 |
| Magnesium stearate | 1 |

### Example 6

### Manufacturing Method and Composition of GIT “Stealth” Encasement

| | % composition |
|---|---|
| Methacrylic acid copolymer type A/B | 12 |
| PEG 600 | 2 |
| water | 5 |
| Talc | 8 |
| Titanium dioxide | 5 |
| Pigment | 8 |
| Ethanol | 60 |

Eudragit L/S was added to ethanol using a silverson high shear mixer (solution A). Secondly, PEG 600 was added to water using a propeller stirrer (solution B). Talc, pigment and titanium dioxide were added to ethanol (suspension C) using a propeller mixer. Solution B was added into suspension C and mixed vigorously. This mixture was then added to solution A under high shear mixing conditions to obtain the GIT “stealth” encasement.

### Example 7

### Bupropion ER

| | % composition |
|---|---|
| Bupropion | 39 |
| Hydroxypropyl methylcellulose | 35 |
| Ethylcellulose | 5 |
| Hydroxyethylcellulose | 5 |
| Lactose | 10 |
| Microcrystalline cellulose | 5 |
| Silicone dioxide | 0.6 |
| Caprylocaproyl or oleoyl or | 5 |
| linoleoyl macrogolglycerides | |
| Magnesium stearate | 0.4 |

### Example 8

### Buspirone Hydrochloride ER 20 mg

| | % composition |
|---|---|
| Buspirone HCl | 5 |
| Hydroxypropyl methylcellulose | 35 |
| Ethylcellulose | 6 |
| Hydroxyethylcellulose | 15 |
| Lactose | 30 |
| Microcrystalline cellulose | 8 |
| Magnesium stearate | 1 |

### Example 9

### Tramadol Hydrochloride ER 200 mg

| | % composition |
|---|---|
| Tramadol hydrochloride | 37 |
| Hydroxypropyl methylcellulose | 33 |
| Ethylcellulose | 5 |
| Hydroxyethylcellulose | 5 |
| Lactose | 10 |
| Microcrystalline cellulose | 8 |
| Magnesium stearate | 1 |
| Talc | 1 |

### Example 10

### Topiramate 200 mg

| | Formulation A |
|---|---|
| Component | % by weight |
| Topiramate | 50% |
| Methocel K100LV CR Premium | 7.5% |
| (Hydroxypropylmethylcellulose) | |
| Methocel K15M Premium | 7.5% |
| (Hydroxypropylmethylcellulose) | |
| Natrosol 250 HHX | 3.5% |
| (Hydroxyethylcellulose) | |
| Flowlac 100 | 25.5% |
| (Lactose) | |
| Ethocel 100FP Premium | 5% |
| (Ethylcellulose) | |
| Magnesium Stearate | 1% |

Topiramate is blended with Methocel K100LV CR Premium, Methocel K15M Premium, Natrosol 250HHX and Flowlac in a Diosna P1-6 high shear mixer for 5 minutes with the chopper motor set at 600 rpm and the mixer motor set at 400 rpm. The blend is granulated with 2-propanol for 5 minutes and the granules are dried in a Casburt laminar flow drying oven at a temperature of 40° C. for 18 h and screened through a 800 μm screen. The granules and the Ethocel 100FP are blended in a V-type PK Blendmaster with a mixing time of 5 minutes with set speeds for the blender shell and intensifier bar. Magnesium stearate is added to the blend and the mixture is further blended for 1.5 min with set speed for the blender shell and the intensifier bar turned off. The blend is compressed into tablets using a 9 mm normal concave round tooling in a Riva Piccola Bi-layer tablet press.

Using a similar procedure, tablets with the following compositions are prepared.

| | Formula- | Formulation | Formulation |
|---|---|---|---|
| | tion B % | C | D |
| Component | by weight | % by weight | % by weight |
| Topiramate | 50% | 50% | 50% |
| Methocel K100LV CR Premium | 15% | 0% | 0% |
| (Hydroxypropylmethylcellulose) | | | |
| Methocel K15M Premium | 0% | 15% | 15% |
| (Hydroxypropylmethylcellulose) | | | |
| Natrosol 250 HHX | 3.5% | 3.5% | 3.5% |
| (Hydroxyethylcellulose) | | | |
| Flowlac 100 | 25.5% | 25.5% | 10.5% |
| (Lactose) | | | |
| Poloxamer F127 | 0% | 0% | 15% |
| (Surfactant) | | | |
| Ethocel 100FP Premium | 5% | 5% | 5% |
| (Ethylcellulose) | | | |
| Magnesium Stearate | 1% | 1% | 1% |
| (Lubricant) | | | |

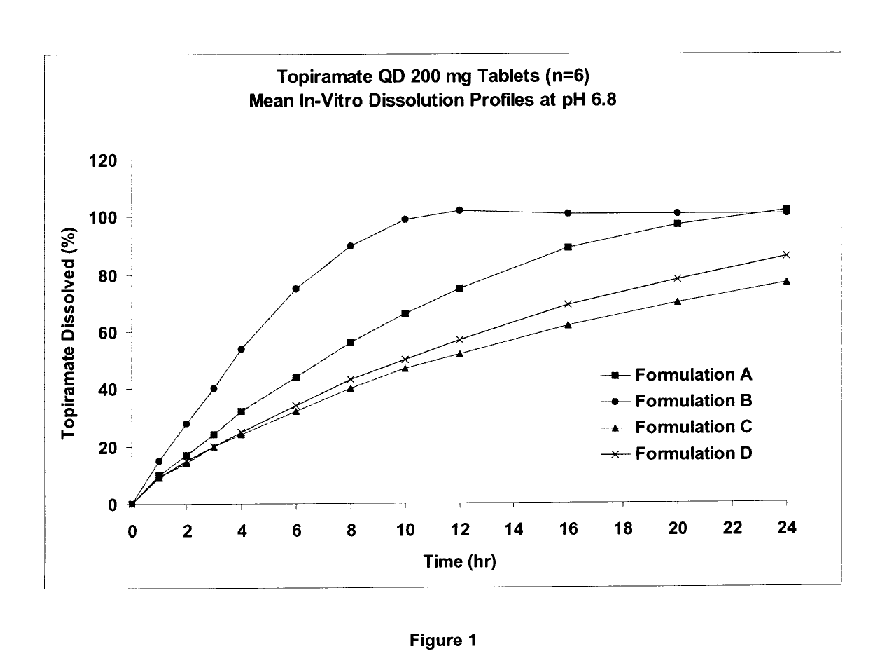

FIG. 1 shows the dissolution profiles of formulations A, B, C and D, measured using the stationary basket method (USP Type 2) in 500 mL of phosphate buffer, pH 6.8, at a paddle speed of 50 rpm at 37° C.±0.5° C. Under these conditions, formulations A, B, C and D exhibit a dissolution profile characterized by the following equation:

y=100−100*e^{(-a*x}^{^{b}})

where:

y=% dissolution;

x=sampling time;

a=scale parameter which ranges from about 0.06 to about 0.12;

b=shape parameter which ranges from about 0.9 to about 1.5; and

100=the cumulative percentage of the topiramate released at time infinity.

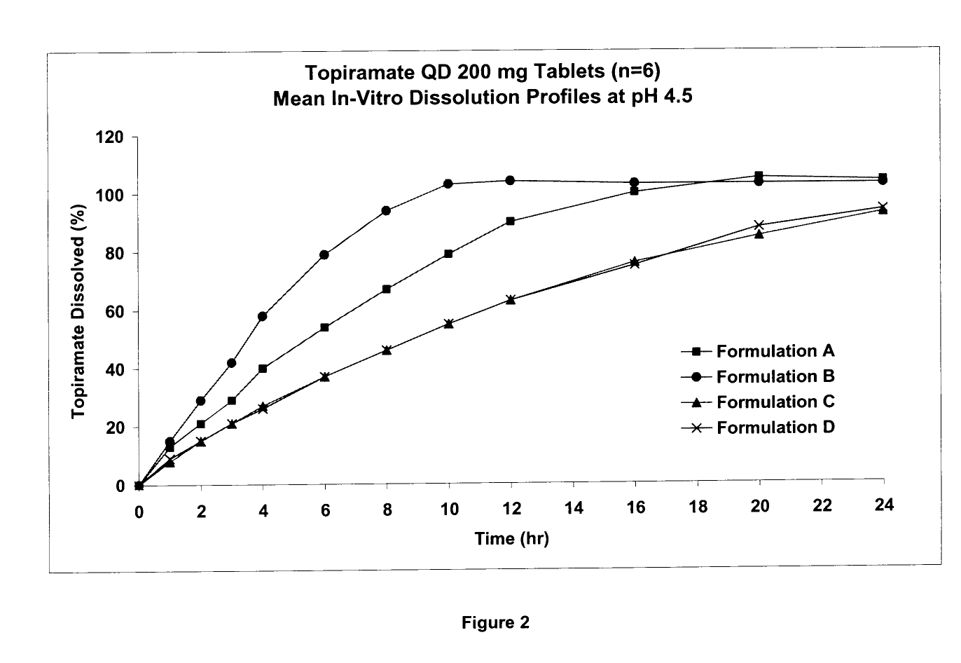

FIG. 2 shows the dissolution profiles of formulations A, B, C and D, measured using the stationary basket method (USP Type 2) in 500 mL of USP acetate buffer, pH 4.5, at a paddle speed of 50 rpm at 37° C.±0.5° C. Under these conditions, formulations A, B, C and D exhibit a dissolution profile characterized by the following equation:

y=100−100*e^{(-a*x}^{^{b}})

where:

y=% dissolution;

x=sampling time;

a=scale parameter which ranges from about 0.06 to about 0. 12;

b=shape parameter which ranges from about 1.1 to about 1.6; and

100=the cumulative percentage of the topiramate released at time infinity.

### Pharmacokinetic Study of Topiramate 200 mg Tablets

The study evaluates the bioavailability of 200 mg topiramate formulations A, B, C and D, described above, relative to Topamax® administered as 100 mg b.i.d. under single dose fasting conditions. The study followed a three-period, randomized, open-label, single dose crossover design with two separate randomization schemes.

The study population consisted of 36 subjects (18 male, 18 female) randomly assigned to two groups of 18 subjects each (9 male, 9 female). In Group 1, 15 subjects completed the study and in Group 2, 13 subjects completed the study.

Group 1 received the following treatments:
Treatment A: Oral dose of one Topiramate 200 mg tablet, Formulation A, with 240 mL of water after a 10 hour overnight fast.
Treatment B: Oral dose of one Topiramate 200 mg tablet, Formulation B, with 240 mL of water after a 10 hour overnight fast.
Treatment E: Oral dose of one Topamax® 100 mg tablet with 240 mL of water at 0 hour after a 10 hour overnight fast; followed by another 100 mg tablet with 240 mL of water 12 hours later, after a 2 hour fast.

Group 2 received the following treatments:
Treatment C: Oral dose of one Topiramate 200 mg tablet, Formulation C, with 240 mL of water after a 10 hour overnight fast.
Treatment D: Oral dose of one Topiramate 200 mg tablet, Formulation D, with 240 mL of water after a 10 hour overnight fast.
Treatment E: Oral dose of one Topamax® 100 mg tablet with 240 mL of water at 0 hour after a 10 hour overnight fast; followed by another 100 mg tablet with 240 mL of water 12 hours later, after a 2 hour fast.

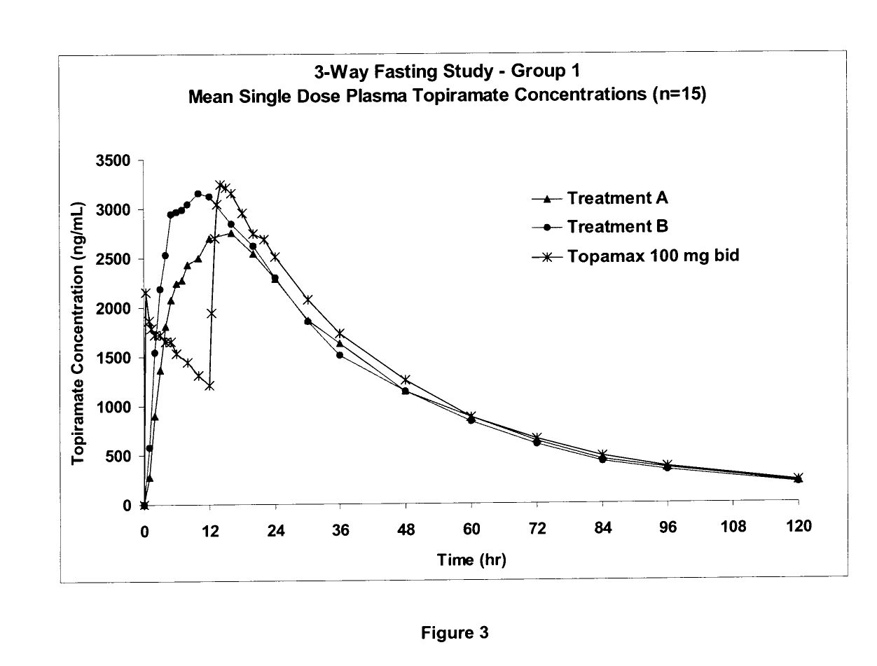

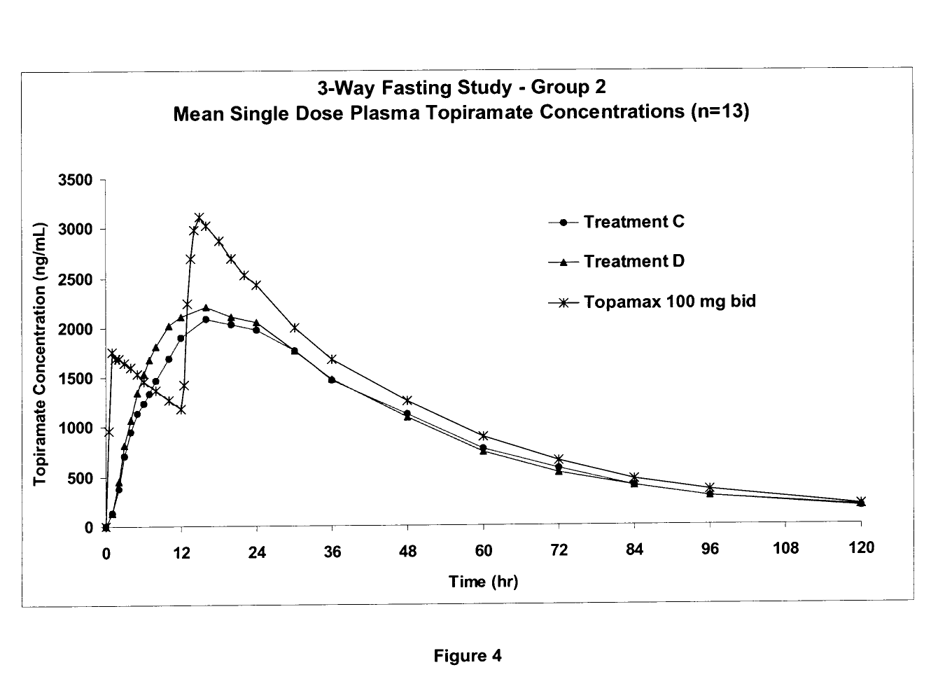

The study periods were separated by a 20 day washout period. Pharmacokinetic and statistical analyses carried out on plasma topiramate concentration-time data from each group give the mean plasma concentration-time plots shown in FIG. 3 (Group 1) and FIG. 4 (Group 2). Mean pharmacokinetic parameters and summary statistics are shown in Tables 1 and 2 below. The results show that, in at least one embodiment, controlled release matrix tablets comprising topiramate or a pharmaceutically acceptable salt thereof according to the present invention exhibit bioequivalence to Topamax®.

TABLE 1
Mean (±SD) Pharmacokinetic Parameters for Topiramate
200 mg Tablets (Group 1 n = 15; Group 2 n = 13)

| | Group 1 | | | Group 2 | | |
|---|---|---|---|---|---|---|
| | | | Topamax ® | | | Topamax ® |
| | | | 100 mg, | | | 100 mg, |
| Parameter | Treatment A | Treatment B | b.i.d. | Treatment C | Treatment D | b.i.d. |
| AUC₀₋ₜ | 131935 ± 21045 | 137831 ± 20555 | 135735 ± 25173 | 107044 ± 38432 | 112486 ± 44050 | 131346 ± 31788 |
| (ng · h/mL) | | | | | | |
| AUC_{0-∞} | 141640 ± 23496 | 147648 ± 20734 | 146884 ± 27758 | 108893 ± 35472 | 120848 ± 45530 | 139682 ± 34715 |
| (ng · h/mL) | | | | | | |
| Cₘₐₓ | 2872.2 ± 555.9 | 3327.3 ± 652.5 | 3720.9 ± 626.9 | 2276.4 ± 786.4 | 2410 ± 827.3 | 3214.4 ± 624.8 |
| (ng/mL) | | | | | | |
| Tₘₐₓ (h) | 16.0 | 10.0 | 1.5 | 16.0 | 16.0 | 2.0 |
| | (6.0-20.0) | (5.0-16.0) | (0.5-4.0) | (5.0-48.0) | (5.0-24.0) | (1.5-8.0) |
| T_{1/2} (h) | 29.9 ± 4.3 | 27.4 ± 4.2 | 29.0 ± 5.5 | 33.1 ± 8.9 | 31.9 ± 8.2 | 28.0 ± 4.0 |

TABLE 2
Comparison of Pharmacokinetic Parameters of Topiramate 200 mg Tablets
with Topamax ® 100 mg, b.i.d. (% Ratio; (90% Confidence Intervals))

| | Group 1 | | Group 2 | |
|---|---|---|---|---|
| Parameter | Treatment A | Treatment B | Treatment C | Treatment D |
| AUC₀₋ₜ | 97.52 | 102.2 | 77.89 | 81.27 |
| | (92.18-103.07) | (96.47-107.91) | (66.11-94.87) | (68.31-97.46) |
| AUC_{0-∞} | 97.70 | 99.27 | 79.56 | 82.79 |
| | (91.19-102.63) | (94.93-106.81) | (67.32-96.47) | (69.74-99.99) |
| Cₘₐₓ | 76.86 | 89.01 | 67.99 | 72.12 |
| | (72.05-82.06) | (83.38-94.93) | (59.62-79.64) | (62.81-83.93) |

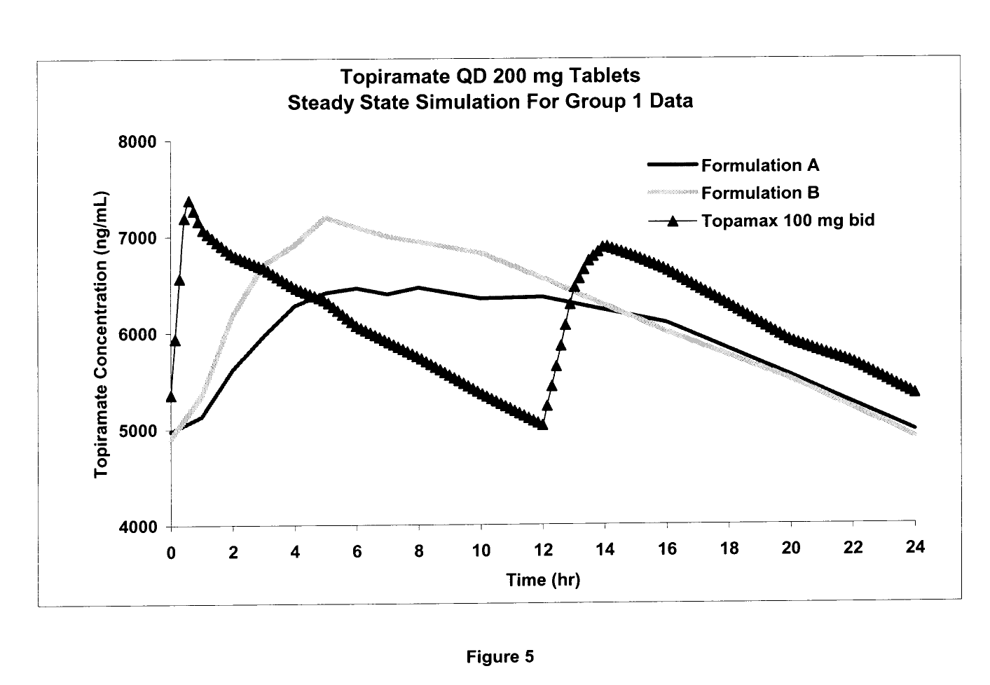

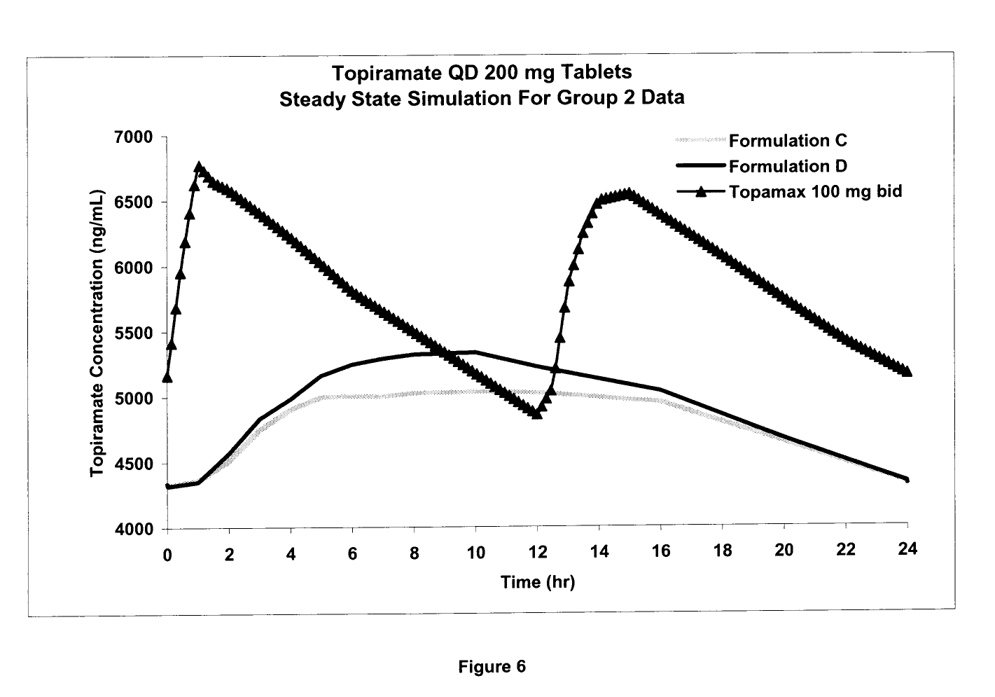

The results of a steady state simulation, performed to predict plasma profiles under repeated drug administration for 14 days, are shown in FIG. 5 (Group 1) and FIG. 6 (Group 2). Values of predicted pharmacokinetic parameters relative to Topamax® are shown in Table 3.

TABLE 3
Comparison of Predicted Pharmacokinetic Parameters
(Steady State Simulation) of Topiramate 200 mg Tablets
with Topamax ® 100 mg, b.i.d. (% Ratio)

| | Group 1 | | Group 2 | |
|---|---|---|---|---|
| Parameter | Treatment A | Treatment B | Treatment C | Treatment D |
| AUC | 97 | 101 | 82 | 85 |
| Cₘₐₓ | 88 | 98 | 74 | 79 |
| Cₘᵢₙ | 93 | 92 | 84 | 84 |

### In Vitro/In Vivo Correlation

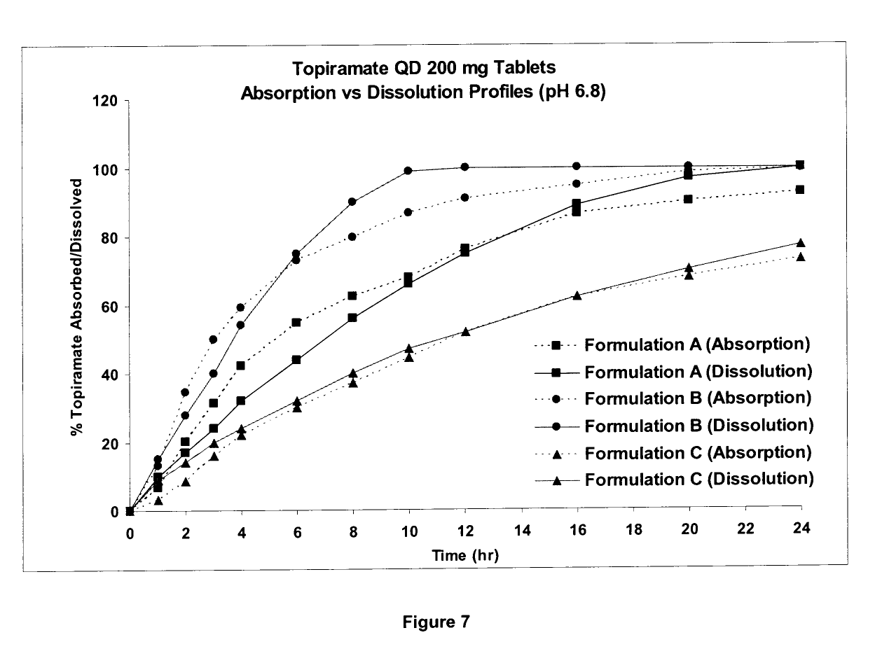

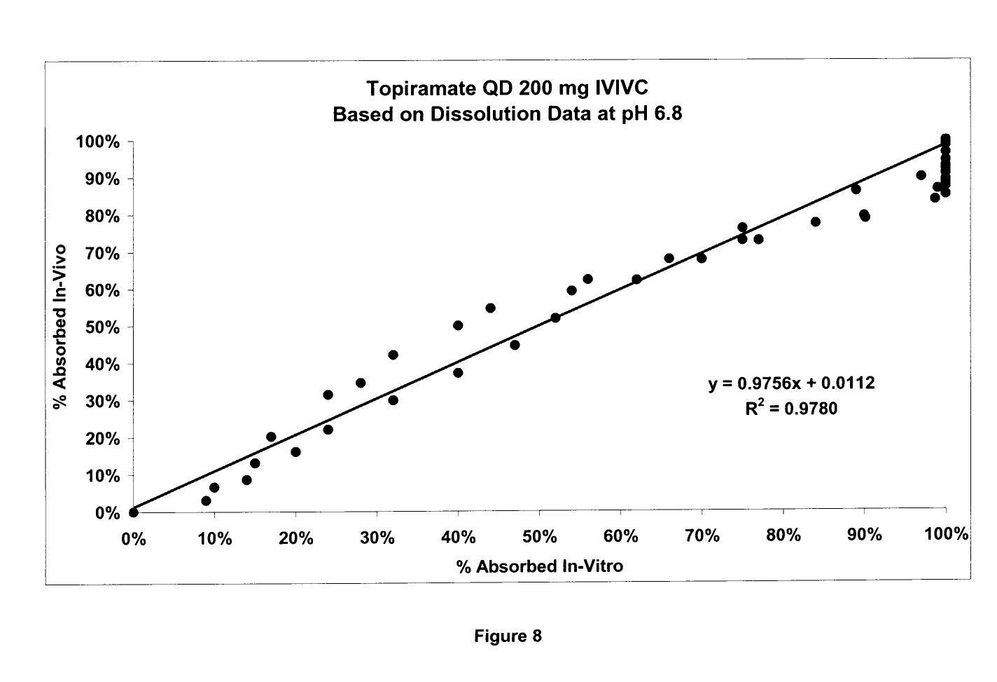

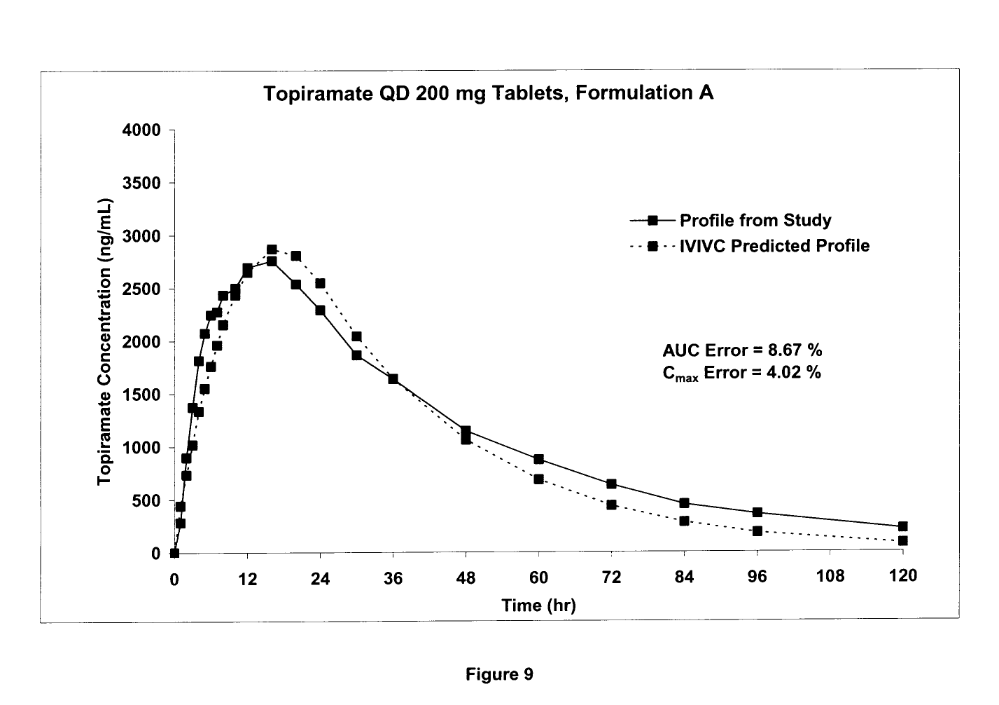

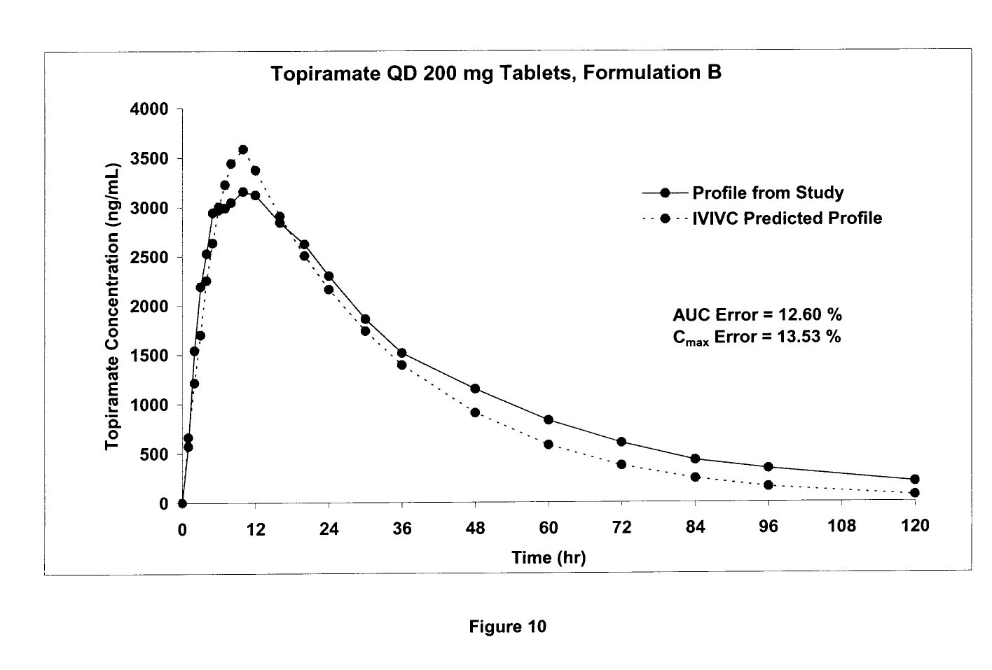

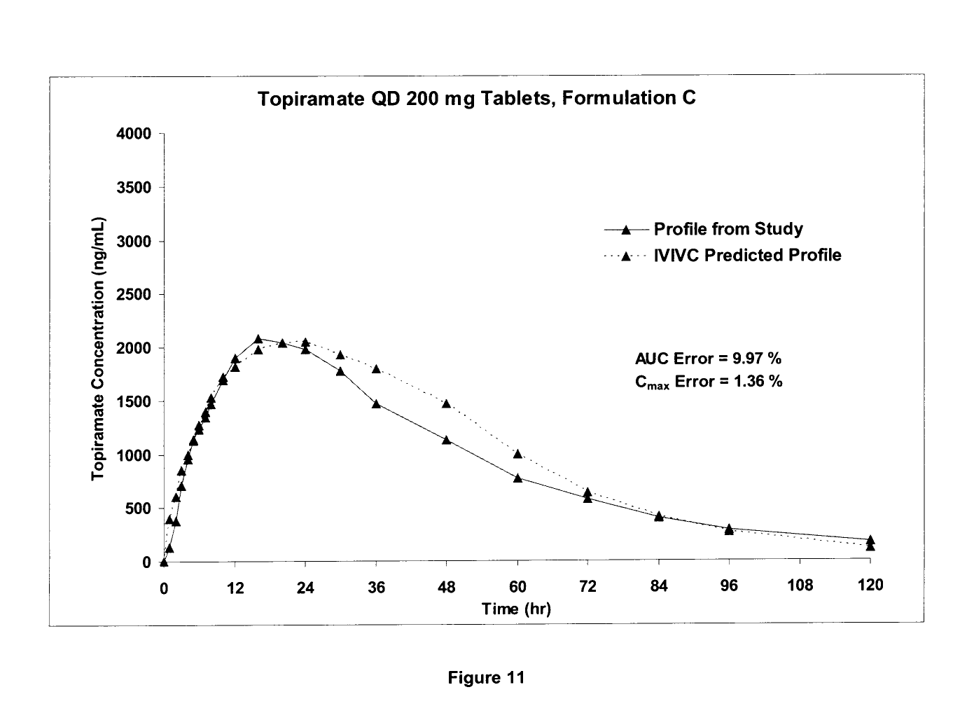

An in vitro/in vivo correlation (IVIVC) is obtained by generating the in vivo absorption profiles of formulations A, B and C by numerical deconvolution. A plot comparing the absorption profiles to the in vitro dissolution profiles obtained at pH 6.8 is shown in FIG. 7. The in vivo absorption data are then correlated with the corresponding in vitro dissolution data, using a linear “Type A” correlation, represented by the function y=mx+b, wherein m=0.9756 and b=0.0112, as shown in FIG. 8. The R² value is calculated to be 0.9780. The IVIVC is internally validated by comparing the actual plasma topiramate concentration-time profiles to those predicted from the in vitro dissolution rates for Formulations A, B and C as shown in FIGS. 9-11. The predictability of the IVIVC is determined by the absolute relative percent errors in AUC and Cₘₐₓ between the actual profile and that predicted by the IVIVC. For AUC, the Maximum Absolute Percent Prediction Error (MAPPE) and Average Absolute Percent Prediction Error (AAPPE) are 12.60% and 10.42% respectively. For Cₘₐₓ, the MAPPE and AAPPE are 13.53% and 6.30% respectively.

Although preferred embodiments have been described herein in detail, it is understood by those skilled in the art that variations may be made thereto without departing from the scope of the invention or the spirit of the appended claims.

1. A controlled release matrix tablet comprising:
(a) topiramate or a pharmaceutically acceptable salt thereof;
(b) a first intelligent polymer component; and
(c) a second intelligent polymer component having opposite wettability characteristics to said first intelligent polymer component;
wherein the first intelligent polymer component is more hydrophobic than the second intelligent polymer component; and wherein the first and second intelligent polymer components constitute a substantially homogeneous matrix, wherein the topiramate or pharmaceutically acceptable salt thereof is substantially homogeneously dispersed in the substantially homogeneous matrix.

2. The controlled release matrix tablet of claim 1, wherein the first and second intelligent polymers have the following single and three-component solubility parameters (MPa^{0.5}) calculated using the group contribution method:

| | δ | δₜ | δ_{d} | δₚ | δₕ | δ₋ₐ |
|---|---|---|---|---|---|---|
| First intelligent polymer | 15-25 | 14-24 | 12-17 | 2-7 | 5-15 | 6-13 |
| Second intelligent polymer | 18-50 | 18-45 | 12-17 | 2-8 | 12-20 | 13-20 |

wherein δ is the conventional Hildebrand parameter, ₜ= total, _{d}= dispersion, ₚ= polar, ₕ= hydrogen bond and ₐ= association interactions.

3. The controlled release matrix tablet of claim 1, wherein the first intelligent polymer component is present in an amount of not less than about 5% by weight.

4. The controlled release matrix tablet of claim 1 wherein the first intelligent polymer component is ethylcellulose.

5. The controlled release matrix tablet of claim 1 wherein the second intelligent polymer component is present in an amount of from about 15% to about 50% by weight.

6. The controlled release matrix tablet of claim 1 wherein the second intelligent polymer component is a mixture of hydroxyethylcellulose and hydroxypropylmethylcellulose.

7. The controlled release matrix tablet of claim 1 further comprising at least one pharmaceutically acceptable excipient.

8. The controlled release matrix tablet of claim 1, wherein the composition further comprises about 10% to about 70% by weight of at least one channeling agent.

9. The controlled release matrix tablet of claim 8, wherein the at least one channeling agent is lactose.

10. The controlled release matrix tablet of claim 1, wherein the composition further comprises about 0.1% to about 5% by weight of at least one lubricant.

11. The controlled release matrix tablet of claim 10, wherein the at least one lubricant is magnesium stearate.

12. The controlled release matrix tablet of claim 1, wherein said controlled release matrix tablet further comprises about 0.25% to about 5% by weight of at least one glidant.

13. The controlled release matrix tablet of claim 12, wherein the glidant is silicon dioxide.

14. The controlled release matrix tablet of claim 1 wherein said controlled release matrix tablet further comprises up to about 15% by weight of at least one surface active agent.

15. The controlled release matrix tablet of claim 14, wherein said surface active agent is a block copolymer of polyoxyethylene and polyoxypropylene.

16. The controlled release matrix tablet of claim 1 wherein said controlled release matrix tablet is free of a surface active agent.

17. The controlled release matrix tablet of claim 1, wherein said controlled release matrix tablet further comprises about 5% to about 30% of at least one compression enhancer.

18. The controlled release matrix tablet of claim 17, wherein said compression enhancer is microcrystalline cellulose.

19. The controlled release matrix tablet of claim 1 comprising:
(a) from about 0.5% to about 70% by weight of topiramate or a pharmaceutically acceptable salt thereof;
(b) not less than about 5% by weight ethylcellulose; and
(c) from about 15% to about 50% by weight of a mixture of hydroxyethylcellulose and hydroxypropylmethylcellulose, wherein the ratio of hydroxyethylcellulose to hydroxypropylmethylcellulose is from about 1:100 to about 100:1.

20. The controlled release matrix tablet of claim 1 comprising:
(a) about 50% by weight of topiramate or a pharmaceutically acceptable salt thereof;
(b) about 5% by weight of ethylcellulose;
(c) about 3.5% by weight of hydroxyethylcellulose and about 15% by weight of hydroxypropylmethylcellulose.

21. The controlled release matrix tablet of claim 1 wherein the tablet is uncoated.

22. The controlled release matrix tablet of claim 1 wherein the tablet is coated with one or more coats.

23. The controlled release matrix tablet of claim 22 wherein the tablet is coated with a coating composition which undergoes gradual dissolution in the gastrointestinal (GI) system.

24. The controlled release matrix tablet of claim 23 wherein the coating composition which undergoes gradual dissolution in the gastrointestinal (GI) system comprises an anionic copolymer of methacrylic acid and methyl methacrylate.

25. A controlled release matrix tablet comprising topiramate or a pharmaceutically acceptable salt thereof wherein the tablet is bioequivalent to a reference formulation of topiramate or a pharmaceutically acceptable salt thereof.

26. A controlled release matrix tablet comprising topiramate or a pharmaceutically acceptable salt thereof wherein the tablet exhibits an in vitro/in vivo correlation.

27. A controlled release matrix tablet comprising topiramate or a pharmaceutically acceptable salt thereof wherein the tablet exhibits a dissolution profile at pH 6.8 or at 4.5 such that after about 1 hour, no more than about 15% of the topiramate or pharmaceutically acceptable salt thereof is released; after about 2 hours, from about 5% to about 35% of the topiramate or pharmaceutically acceptable salt thereof is released; after about 4 hours, from about 20% to about 60% of the topiramate or pharmaceutically acceptable salt thereof is released; after about 8 hours, from about 35% to about 95% of the topiramate or pharmaceutically acceptable salt thereof is released; after about 12 hours, no less than about 50% of the topiramate or pharmaceutically acceptable salt thereof is released; and after about 16 hours, no less than about 60% of the topiramate or pharmaceutically acceptable salt thereof is released.

28. The controlled release matrix tablet of claim 27 wherein the tablet exhibits a dissolution profile at pH 6.8 such that after about 1 hour, no more than about 15% of the topiramate or pharmaceutically acceptable salt thereof is released; after about 2 hours, from about 15% to about 35% of the topiramate or pharmaceutically acceptable salt thereof is released; after about 4 hours, from about 30% to about 60% of the topiramate or pharmaceutically acceptable salt thereof is released; after about 8 hours, from about 55% to about 90% of the topiramate or pharmaceutically acceptable salt thereof is released; after about 12 hours, no less than about 75% of the topiramate or pharmaceutically acceptable salt thereof is released; and after about 16 hours, no less than about 85% of the topiramate or pharmaceutically acceptable salt thereof is released.

29. The controlled release matrix tablet of claim 27 wherein the tablet exhibits a dissolution profile at pH 4.5 such that after about 1 hour, no more than about 15% of the topiramate or pharmaceutically acceptable salt thereof is released; after about 2 hours, from about 20% to about 30% of the topiramate or pharmaceutically acceptable salt thereof is released; after about 4 hours, from about 40% to about 60% of the topiramate or pharmaceutically acceptable salt thereof is released; after about 8 hours, from about 65% to about 95% of the topiramate or pharmaceutically acceptable salt thereof is released; and after about 12 hours, no less than about 90% of the topiramate or pharmaceutically acceptable salt thereof is released.

30. A method of treating seizures in a patient in need thereof, the method comprising administering the controlled release tablet of claim 1 to the patient.

31. A method of treating or preventing migraine in a patient in need thereof, the method comprising administering the controlled release tablet of claim 1 to the patient.

32. A method of treating obesity in a patient in need thereof, the method comprising administering the controlled release tablet of claim 1 to the patient.

33. A method of treating alcohol, cocaine and/or tobacco dependence in a patient in need thereof, the method comprising administering the controlled release tablet of claim 1 to the patient.

34. A method of treating bipolar disorder in a patient in need thereof, the method comprising administering the controlled release tablet of claim 1 to the patient.
